(19) United States
(12) Patent Application Publication
(10) Pub. No.: US 2009/0143429 A1
(43) Pub. Date: Jun. 4, 2009

(54) Quinoline Derivatives as Neurokinin Receptor Antagonists

(76) Inventors: James Michael Crawforth, Kent (GB); Brian John Williams, Great Dunmow (GB)

Correspondence Address: MERCK AND CO., INC, P O BOX 2000, RAHWAY, NJ, 07065-0907, US

(21) Appl. No.: 12/225,931

(22) PCT Filed: Jul. 25, 2006
(86) PCT No.: PCT/GB2006/050221

§ 371 Date: Feb. 11, 2009

(30) Foreign Application Priority Data
Jul. 29, 2005 (GB) 0515580.9

(51) Int. Cl. A61K 31/4709; C07D 401/06; A61P 25/30

(52) U.S. Cl. 514/314; 546/169

(57) ABSTRACT

The present invention relates to substituted quinoline derivatives of Formula (I); wherein hal, n, A, formula (a), R¹, R², R³, R⁴, R⁵ and R⁶ are defined herein, pharmaceutical compositions comprising them and their use in treating diseases mediated by neurokinin-2 and/or neurokinin-3 (NK-3) receptors. These compounds can thus be used in methods of treatment to suppress and treat such disorders.

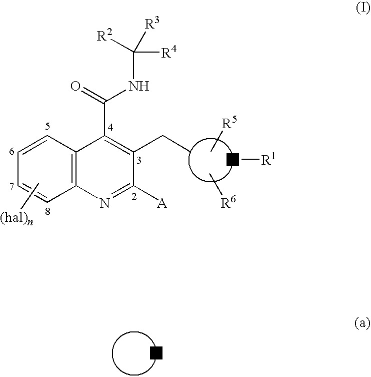

The present invention relates to substituted quinoline-4-carboxamide derivatives, pharmaceutical compositions comprising them and their use in treating diseases mediated by neurokinin-2 (NK-2) and/or neurokinin-3 (NK-3) receptors. These compounds can thus be used in methods of treatment to suppress and treat such disorders.

Background information on NK-3 receptor antagonists can be found in literature reviews such as Giardina and Raveglia, Exp. Opin. Ther. Patents (1997) 2(4): 307-323 and Giardina et al., Exp. Opin. Ther. Patents (2000) 10(6): 939-960. These references also contain pertinent information on preclinical validation of therapies that can be treated with NK-3 antagonists.

Certain quinoline derivatives have already been disclosed as NK-3 receptor antagonists. For instance, published International patent applications WO 2005/014575, WO 2005/000247, WO 2004/066951, WO 2004/066950, WO 2004/050627, WO 2004/050626 (all SmithKline Beecham Corporation), WO 02/083664, WO 02/083663, WO 02/083645, WO 02/44165, WO 02/44154, WO 02/43734, WO 02/38548, WO 02/38547 (all GlaxoSmithKline S.P.A.), WO 00/64877, WO 00/58307 (both Neurogen Corporation), WO 00/31038, WO 00/31037, WO 98/52942, WO 97/21680, WO 97/19928, WO 97/19926, WO 96/02509 and WO 95/32948 (all SmithKline Beecham S.P.A.) disclose quinoline-4-carboxamide derivatives as NK-3 receptor antagonists.

The present invention thus provides a compound of formula (I):

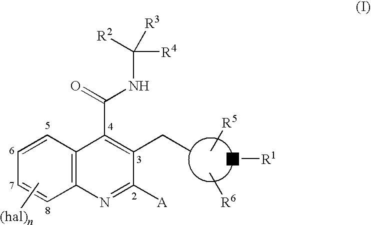

or a pharmaceutically acceptable salt thereof,
wherein
hal is fluorine, chlorine, bromine or iodine;
n is 0, 1 or 2, and when n is 2, the two hal atoms may be the same or different;
A is phenyl or thiophenyl, optionally substituted by 1 to 3 halogen atoms;

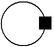

is a C-linked azetidinyl, pyrrolidinyl or piperidinyl ring, optionally bridged by a C₁₋₃alkylene group, and optionally fused to phenyl;
R¹ is hydrogen, C₁₋₆alkyl, C₂₋₆alkenyl, C₂₋₆alkynyl, C₃₋₈cycloalkyl, C(O)C₁₋₆alkyl, C(O)OC₁₋₆alkyl, C(O)O(CH₂)₀₋₃aryl, S(O)₂C₁₋₆alkyl, heteroaryl or Het, where C₃₋₈cycloalkyl, aryl, heteroaryl and Het are optionally substituted by C₁₋₆alkyl, and where Het is a heteroaliphatic ring of 4 to 6 ring atoms, which ring contains 1 or 2 heteroatoms selected from N, O and S or a group S(O), S(O)₂, NH or NC₁₋₄alkyl;
R² is hydrogen, C₁₋₆alkyl, C₂₋₆alkenyl, C₂₋₆alkynyl or C₃₋₈cycloalkyl;
R³ is C₁₋₆alkyl, C₂₋₆alkenyl, C₂₋₆alkynyl, (CH₂)₀₋₃C₃₋₈cycloalkyl or (CH₂)₀₋₃phenyl, optionally substituted by 1 to 3 halogen atoms;
R⁴ is hydrogen, C₁₋₆alkyl, C₂₋₆alkenyl, C₂₋₆alkynyl or C₃₋₈cycloalkyl;
or R² and R⁴ are linked together to form a C₃₋₈cycloalkyl or Het group as hereinbefore defined;
R⁵ is hydrogen, C₁₋₆alkyl, C₂₋₆alkenyl, C₂₋₆alkynyl, C₃₋₈cycloalkyl or oxo;
R⁶ is hydrogen, hydroxy or oxo; with the proviso that when R⁶ is hydroxy it is not attached to the carbon atom adjacent to the ring N atom;
or R¹ and R⁵ together form a nitrogen-containing heteroaliphatic ring, optionally containing one further N or O atom, and optionally substituted by C₁₋₆alkyl.

In one embodiment of the present invention, there is provided a compound of formula (Io):

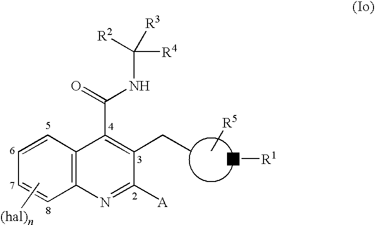

or a pharmaceutically acceptable salt thereof,
wherein
hal is fluorine, chlorine, bromine or iodine;
n is 0, 1 or 2, and when n is 2, the two hal atoms may be the same or different;
A is phenyl or thiophenyl, optionally substituted by 1 to 3 halogen atoms;

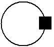

is a C-linked azetidinyl, pyrrolidinyl or piperidinyl ring, optionally bridged by a C₁₋₃alkylene group, and optionally fused to phenyl;
R¹ is hydrogen, C₁₋₆alkyl, C₂₋₆alkenyl, C₂₋₆alkynyl, C₃₋₈cycloalkyl or Het, where C₃₋₈cycloalkyl and Het are optionally substituted by C₁₋₆alkyl, and where Het is a heteroaliphatic ring of 4 to 6 ring atoms, which ring contains 1 or 2 heteroatoms selected from N, O and S or a group S(O), S(O)₂, NH or NC₁₋₄alkyl;
R² is hydrogen, C₁₋₆alkyl, C₂₋₆alkenyl, C₂₋₆alkynyl or C₃₋₈cycloalkyl;
R³ is C₁₋₆alkyl, C₂₋₆alkenyl, C₂₋₆alkynyl, (CH₂)₀₋₃C₃₋₈cycloalkyl or (CH₂)₀₋₃phenyl, optionally substituted by 1 to 3 halogen atoms;
R⁴ is hydrogen, C₁₋₆alkyl, C₂₋₆alkenyl, C₂₋₆alkynyl or C₃₋₈cycloalkyl;
or R² and R⁴ are linked together to form a C₃₋₈cycloalkyl or Het group as hereinbefore defined;
R⁵ is hydrogen, C₁₋₆alkyl, C₂₋₆alkenyl, C₂₋₆alkynyl or C₃₋₈cycloalkyl;
or R¹ and R⁵ together form a nitrogen-containing heteroaliphatic ring, optionally containing one further N or O atom, and optionally substituted by C₁₋₆alkyl.

In another embodiment of the present invention, hal is fluorine, chlorine or bromine. Preferably, hal is fluorine.

In another embodiment of the present invention, n is 1 or 2. Preferably, n is 1.

When n is 1 or 2, preferably one hal group is at the 5-, 7- or 8-position of the quinolinyl ring system. More preferably, one hal group is at the 8-position of the quinolinyl ring system.

In another embodiment of the present invention, A is phenyl, optionally substituted by 1 or 2 halogen atoms. Preferably, A is phenyl.

In another embodiment of the present invention,

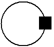

is a C-linked pyrrolidinyl (i.e. 2- or 3-pyrrolidinyl) or piperidinyl (i.e. 2-, 3- or 4-piperidinyl) ring. Preferably,

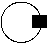

is a C-linked piperidinyl ring. More preferably,

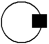

is 4-piperidinyl.

In another embodiment of the present invention, R¹ is hydrogen, C₁₋₆alkyl, C(O)C₁₋₄alkyl, C(O)OC₁₋₄alkyl, C(O)O(CH₂)₀₋₁phenyl, S(O)₂C₁₋₄alkyl, heteroaryl or Het, where Het is as hereinbefore defined and optionally substituted by C₁₋₆alkyl. Preferably, R¹ is hydrogen, C₁₋₄alkyl, C(O)C₃₋₄alkyl, C(O)OC₃₋₄alkyl, C(O)O(CH₂)₀₋₁phenyl, S(O)₂C₁₋₂alkyl, pyrimidyl or a heteroaliphatic ring of 5 or 6 ring atoms, which ring contains either an O or S atom or a group S(O), S(O)₂, NH or NC₁₋₄alkyl, and which ring is optionally substituted by C₁₋₆alkyl. More preferably, R¹ is 3-tetrahydrofuranyl or 3- or 4-tetrahydropyranyl, optionally substituted by C₁₋₆alkyl. Most preferably, R¹ is 3- or 4-tetrahydropyranyl.

Especially, R¹ is 4-tetrahydropyranyl.

In another embodiment of the present invention, R² is C₁₋₆alkyl. Preferably, R² is methyl, ethyl, n-propyl, i-propyl, n-butyl, s-butyl or t-butyl. More preferably, R² is ethyl.

In another embodiment of the present invention, R³ is C₁₋₆alkyl, C₃₋₈cycloalkyl or (CH₂)₀₋₃phenyl. Preferably, R³ is C₁₋₆alkyl, phenyl or CH₂phenyl. More preferably, R³ is phenyl.

In another embodiment of the present invention, R⁴ is hydrogen or C₁₋₆alkyl. Preferably, R⁴ is hydrogen.

In another embodiment of the present invention, R⁵ is hydrogen or C₁₋₆alkyl. Preferably, R⁵ is hydrogen.

In a further embodiment of the present invention, there is provided the compound of formula (Ia):

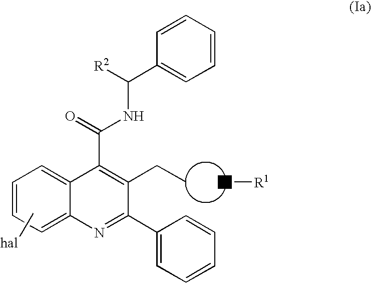

or a pharmaceutically acceptable salt thereof,
wherein hal,

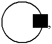

R¹ and R² are as defined in relation to formula (I).

Preferably, hal is fluorine, chlorine or bromine. More preferably, hal is fluorine.

Preferably, hal is at the 7- or 8-position of the quinolinyl ring system. More preferably, hal is at the 8-position of the quinolinyl ring system.

Preferably,

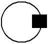

is a C-linked piperidinyl ring, more preferably 3- or 4-piperidinyl, most preferably 4-piperidinyl.

Preferably, R¹ is C₁₋₆alkyl or Het, where Het is as defined in relation to formula (I) and optionally substituted by C₁₋₆alkyl. More preferably, R¹ is a tetrahydropyranyl or thienyl ring, optionally substituted by C₁₋₆alkyl. Most preferably, R¹ is 3- or 4-tetrahydropyranyl. Especially, R¹ is 4-tetrahydropyranyl.

In a further embodiment of the present invention, there is provided the compound of formula (Ib):

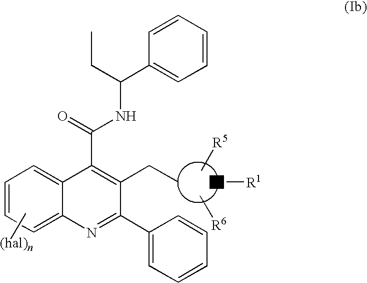

or a pharmaceutically acceptable salt thereof,
wherein hal, n,

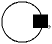

R¹, R⁵ and R⁶ are as defined in relation to formula (I).

Preferably, hal is fluorine, chlorine or bromine. More preferably, hal is fluorine.

Preferably, n is 0 or 1. More preferably, n is 1.

When n is 1, preferably the hal group is at the 8-position of the quinolinyl ring system.

Preferably,

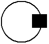

is a C-linked piperidinyl ring. More preferably,

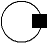

is 3- or 4-piperidinyl.

Preferably, R¹ is hydrogen, C₁₋₆alkyl, C(O)C₁₋₆alkyl, C(O)OC₁₋₆alkyl, C(O)O(CH₂)₀₋₃aryl, S(O)₂C₁₋₆alkyl, heteroaryl or Het. More preferably, R¹ is hydrogen, C₁₋₄alkyl, C(O)C₁₋₄alkyl, C(O)OC₁₋₄alkyl, C(O)O(CH₂)₀₋₃phenyl, S(O)₂C₁₋₄alkyl, pyridinyl or tetrahydropyranyl. Most preferably, R¹ is hydrogen, C₃₋₄alkyl, C(O)C₃₋₄alkyl, C(O)OC₃₋₄alkyl, C(O)O(CH₂)₀₋₁phenyl, S(O)₂C₁₋₂alkyl, pyridinyl or tetrahydropyranyl. Especially, R¹ is hydrogen, propyl, C(O)propyl, C(O)Obutyl, C(O)OCH₂phenyl, S(O)₂CH₃, 2-, 3- or 4-pyridinyl or 2-, 3- or 4-tetrahydropyranyl. More especially, R¹ is hydrogen, ⁱpropyl, C(O)ⁱpropyl, C(O)O^{t}butyl, C(O)OCH₂phenyl, S(O)₂CH₃, 3-pyridinyl or 4-tetrahydropyranyl.

Preferably, R⁵ is hydrogen, C₁₋₆alkyl or oxo. More preferably, R⁵ is hydrogen or oxo. Most preferably, R⁵ is hydrogen.

Preferably, R⁶ is hydrogen.

The present invention includes within its scope solvates of the compounds of formula (I), for example hydrates, and salts thereof.

The present invention also includes within its scope any enantiomers, diasteromers, geometric isomers and tautomers of the compounds of formula (I). It is to be understood that all such isomers and mixtures thereof are encompassed within the scope of the invention.

As used herein, the term “C₁₋₆alkyl” means linear or branched chaln alkyl groups having from 1 to 6 carbon atoms and includes all of the hexyl and pentyl alkyl isomers as well as n-, iso-, sec- and t-butyl, n- and isopropyl, ethyl and methyl.

The term “C₂₋₆alkenyl” means linear or branched chain alkenyl groups having from 2 to 6 carbon atoms and includes all of the hexenyl and pentenyl isomers as well as 1-butenyl, 2-butenyl, 3-butenyl, isobutenyl, 1-propenyl, 2-propenyl, and ethenyl (or vinyl).

The term “C₂₋₆alkynyl” means linear or branched chaln alkynyl groups having from 2 to 6 carbon atoms and includes all of the hexynyl and pentynyl isomers as well as 1-butynyl, 2-butynyl, 3-butynyl, 1-propynyl, 2-propynyl, and ethynyl (or acetylenyl).

The term “alkylene” means that the alkyl group links two separate groups and may be straight or branched. Examples of suitable alkylene groups include ethylene (—CH₂—CH₂—) and propylene (—CH₂—CH₂—CH₂—, CH(CH₃)—CH₂— or CH₂—CH(CH₃)—).

The term “C₃₋₈cycloalkyl” means a cyclic alkane ring having three to eight total carbon atoms (i.e., cyclopropyl, cyclobutyl, cyclopentyl, cyclohexyl, cycloheptyl, or cyclooctyl).

Exemplary compounds of the present invention include those named in the Examples below and their pharmaceutically acceptable salts.

These compounds and those defined by the immediately preceding definitions are useful in therapy, particularly as NK-2 and/or NK-3 antagonists, more particularly as NK-3 antagonists.

The terms “administration of” and or “administering a” compound should be understood to mean providing a compound of the invention to the individual in need of treatment.

The term “subject,” (alternatively referred to herein as “patient”) as used herein refers to an animal, preferably a mammal, most preferably a human, who has been the object of treatment, observation or experiment.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, palmoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate and the like, can be used as the dosage form.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a fmely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chaln aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffm. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffm or mixtures of these. Suitable emulsifying agents may be naturally-occurring gums, for example gum acacia or gum tragacanth, naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require NK-2 and/or NK-3 receptor modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention also provides pharmaceutical compositions comprising a compound of formula (I) or a pharmaceutically acceptable salt thereof and a pharmaceutically acceptable excipient.

Thus, there is provided a compound of formula (I) or a pharmaceutically acceptable salt thereof for use in therapy.

Likewise, there is provided the use of a compound of formula (I) or a pharmaceutically acceptable salt thereof for the manufacture of a medicament for treating a neurokinin-2 and/or neurokinin-3 mediated disease.

There is also disclosed a method of treatment of a subject suffering from a neurokinin-2 and/or neurokinin-3 mediated disease, which comprises administering to that patient a therapeutically effective amount of a compound of formula (I) or a pharmaceutically acceptable salt thereof.

Examples of diseases mediated by neurokinin-2 and/or neurokinin-3 include CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder (GAD), social anxiety disorder (SAD), agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform-diseases, acute psychosis, alcohol psychosis, autism, delirium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); post-traumatic stress disorder; attention deficit hyperactive disorder (ADHD); cognitive impairment (e.g. the treatment of impairment of cognitive functions including attention, orientation, memory (memory disorders, amnesia, amnesic disorders and age-associated memory impairment) and language finction, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, 25 myoclonic seizures, clonic seizures, tonic seizures, tonic clonic seizures and atonic seizures);

psychosexual dysfinction (including inhibited sexual desire (low libido), inhibited sexual arousal or excitement, orgasm dysfinction, inhibited female orgasm and inhibited male orgasm, hypoactive sexual desire disorder (HSDD), female sexual desire disorder (FSDD), and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as Alzheimer's disease, ALS, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophiclateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased, sensitivity, to noxious stimuli (thermal, cold, mechanical hyperalgesia), continuing pain sensation after removal of the stimulation (hyperpathia) or an absence of or deficit in selective sensory pathways (hypoalgesia), pain associated with migraine, and non-cardiac chest pain); certain CNS-mediated disorders such as emesis, irritable bowel syndrome, and non-ulcer dyspepsia; COPD, asthma, cough, gastro-oesophageal reflex induced cough, and exacerbated asthma; urinary incontinence; hypertension; and conditions associated with platelet hyperaggregability such as tissue ulceration, nephrotic syndrome, diabetes, migraine, coronary artery disease, pre-eclampsia, pre-term labour and stroke. Preferably, the compounds of the invention are useful for the treatment of depression; anxiety disorders; phobias; psychosis and psychotic disorders; post-traumatic stress disorder; attention deficit hyperactive disorder (ADHD); withdrawal from abuse of drugs including smoking cessation or reduction in level or frequency of such activities; and irritable bowel syndrome. More preferably, the compounds of the invention are useful for the treatment of depression; anxiety disorders; phobias; and psychosis and psychotic disorders (especially schizophrenia, schizo-affective disorder, and schizophreniform diseases. Most preferably, the compounds of the invention are useful for the treatment of schizophrenia.

The compounds for use in the present invention are generally active in the following tests. They normally have an IC₅₀ of less than 1 μM and preferably less than 100 nM.

Details of the NK-2 receptor and its heterologous expression can be found in Gerard et al., J. Biol. Chem., 265: 20455-20462, 1990 and Huang et al., Biochem., 33: 3007-3013, 1994. The latter paper also contains details of mutant scanning.

Details of the NK-3 receptor and its heterologous expression can be found in Huang et al., BBRC, 1992, 184: 966-972 and Sadowski et al., Neuropeptides, 1993, 24: 317-319.

A membrane preparation is prepared as follows. A 10-layer cell factory is seeded with CHO cells stably expressing NK-3 receptors. The CHO cells are prepared in a triple T175 flask in 11 growth medium which contains Iscore's modified Dulbecco's medium containing 10 ml/l 200 mM L-Glutamine, 10 ml/l penicillin-streptomycin, one vial of hypoxanthine-thymidine 500×/l, 1 mg/ml geneticin and 10% fetal bovine serum (inactivated). The cells are grown for 3 days in an incubator. The medium is washed off and the factory is rinsed twice with 400 ml PBS (Ca, Mg-free). 400 ml enzyme free dissoc. solution (EFDS) is added and the factory is maintained for 10 min at room temperature. The cells are dislodged and the suspension poured into 500 ml centrifuge bottles. The process is repeated with 200 ml EFDS and the mixtures pooled giving 6 bottles in all, which are spun in a centrifuge for 10 min at 2200 rpm.

The supernatants are aspirated and the residual cell pellets are frozen at −80° for 30 min to improve cell lysis and then resuspended in 40 ml Tris with inhibitors per cell factory. The cells are homogenized in 40 ml aliquots with 8 strokes of a glass-teflon grinder at setting 40. The homogenate is transferred to 50 ml centrifuge tubes and placed on a rocker for 15 min at r.t. The homogenate is rehomogenised and held on ice if necessary before being centrifuged again as above.

The supernatant is transferred to Sorvall tubes for an SS-34 roter and held on ice.

40 ml cold Tris with inhibitors is used to resuspend and combine the pellets which are again spun as above. The supernatants are again transferred to Sorvall tubes which, with those above, are spun at 18000 rpm for 20 min.

The supernatants are discarded and the pellets resuspended in a Storage Buffer consisting of 2.50 ml 1M Tris pH7.4, 50 μl 1000×protease inhibitors (4 mg/ml leupeptin (Sigma), 40 mg/ml Bacitracin (Sigma) and 10 mM phosphoranidon (Peninsula) all dissolved in water) plus 0.5 ml 0.5 M MnCl₂ made up to 50 ml with H₂O_{dd}. A 10 ml syringe is used with 20-, 23- and 25-gauge needles sequentially.

A Bradford protein assay in conducted on 2-10 μl aliquots with BSA as standard before 500-1000 μl aliquots are snap-frozen in liquid nitrogen for storage at −80° C.

The membrane binding assay is carried out as follows. The amount of membranes needed to specifically bind ≦10% of ¹²⁵I-NeurokinB is predetermined. The frozen stocks are then diluted to allow addition in 50 μl.

The test compounds are dissolved in DMSO. An automated apparatus (Tecan) is programmed to add 5 μl of compound or DMSO, approximately 100,000 cpm of isotope in 20 μl buffer which is prepared from 50 μM Tris, pH7.5, 150 μM NaCl, bovine serum albumin to 0.02%, and protease inhibitors as in the storage buffer, made up as 0.5M stock, and 175 μl assay buffer (as the storage buffer but containing 5 μM MnCl₂ and without NaCl) into deep well Marsh boxes (Marsh Biomedical Products) in a 96-well format. Excess unlabelled competing peptide is added by hand for non-specific binding as indicated below. The binding reaction is initiated by adding 50 μl of cell membranes. The tubes are incubated with shaking for 1 h at r.t. and filtered on a Tomtec 96 well cell harvester using Mach III filtermats (Tomtec) or using either a Packard 96-well harvester or Tomtec 9600 using Unifilter GF/C (Packard), presoaked in 0.25% polyethyleneimine and washed five times with 1×wash buffer (0.1M.Tris, pH7.4 and 1M NaCl, 1×=100 ml of 10×stock per litre of cold distilled water). If using Unifilter plates, 60 μl Microscint 20 (Packard) is added to each well and the plate is then heat-sealed before counting in a Packard Topcount. Alternatively the filters from the filtermat are placed in 75×100 mm plastic tubes and counted on a Cobra gamma counter.

For the assay, typically 10 μg of membrane is used at 25,000 cpm which is filtered over a Unifilter GF/C presoaked in 0.5% BSA.

Assays for binding at the neurokinin-2 receptor can be carried out in an analogous manner.

The compounds of the present invention can be readily prepared according to the following reaction schemes and examples, or modifications thereof. Starting materials can be made from procedures known in the art or as illustrated. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples.

The compounds of the present invention can be prepared according to the general method shown in Scheme 1:

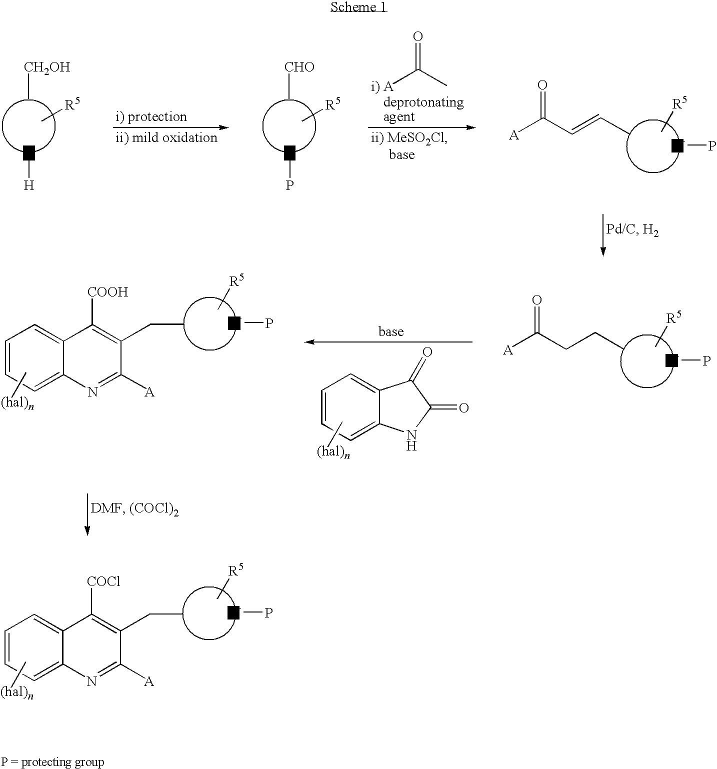

The cyclic amine starting material is protected with a suitable protecting group (e.g. t-butyloxycarbonyl) and the primary alcohol group is then oxidized under mild conditions (e.g. Swern oxidation) to form the corresponding aldehyde. The aldehyde is then reacted with an acetyl derivative in the presence of a deprotonating agent (such as LHMDS) at reduced temperature. The reaction is quenched by the addition of a weak acid (e.g. citric acid). The crude reaction material comprising the β-hydroxy ketone is then submitted to elimination conditions (e.g. using methanesulfonyl chloride) to give the α,β-unsaturated ketone. The ketone is then hydrogenated in the presence of a suitable catalyst (e.g. palladium on carbon) and then reacted with the appropriate isatin derivative under basic conditions (e.g. using KOH) at raised temperature to yield the 4-carboxylic acid quinoline derivative. The carboxylic acid is then reacted with the appropriate reagent, such as oxalyl chloride in the presence of DMF, to provide a reactive carboxylic acid derivative.

In a further aspect, the present invention provides a process for the preparation of a compound of the formula (I) wherein R¹ is hydrogen, which process comprises the reaction of a compound of the formula (II) with an amine of formula (III):

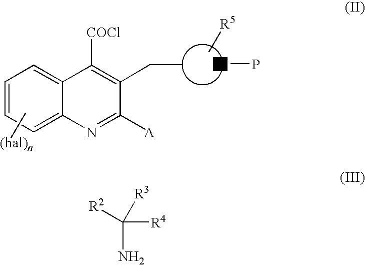

and thereafter removing the protecting group from

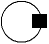

and converting

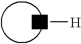

to

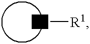

wherein R¹ is other than hydrogen.

The reaction of the compound of formula (II) conveniently takes place in a non-reactive solvent, for example, a haloalkane, such as dichloromethane, at a non-extreme temperature of −20 C to 150 C, preferably −10 C to 50 C.

Compounds of formula (I) can be converted into other compounds of formula (I) using synthetic methodology well known in the art. For instance, the compound of formula (I) where R¹ is hydrogen may be converted into the compound of formula (I) where R¹ is 4-tetrahydropyranyl by reacting the former compound with tetrahydro-4H-pyran-4-one in the presence of a mild reducing agent (such as sodium triacetoxyborohydride) and a mild acid (such as acetic acid) in a suitable solvent (such as a haloalkane, e.g. 1,2-dichloroethane).

Also, the compound of formula (I) where R¹ is hydrogen may be converted into the compound of formula (I) where R¹ is benzyl carboxylate by reacting the former compound with benzylchloroformate in a suitable solvent, such as ethyl acetate.

Furthermore, the compound of formula (I) where R¹ is 4-tetrahydropyranyl may be converted into the compound of formula (I) where R¹ is C₁₋₆alkyl by reacting the former compound with O=C(C₁₋₆alkyl)₂ in the presence of a mild reducing agent (such as sodium triacetoxyborohydride) and a mild acid (such as acetic acid) in a suitable solvent (such as a haloalkane, e.g. 1,2-dichloroethane).

Alternatively, the compound of formula (I) where R¹ is 4-tetrahydropyranyl may be converted into the compound of formula (I) where R¹ is C₁₋₆alkylsulfonyl by reacting the former compound with C₁₋₆alkylsulfonyl chloride in the presence of a base (such as triethylamine) in a suitable solvent (such as a haloalkane, e.g. dichloromethane).

In addition, the compound of formula (I) where R¹ is 4-tetrahydropyranyl may be converted into the compound of formula (1) where R¹ is C-linked pyridinyl by reacting the former compound with pyridine boronic acid in the presence of a catalyst (such as copper (II) acetate).

Also, the compound of formula (I) where R¹ is 4-tetrahydropyranyl may be converted into the compound of formula (I) where R¹ is C₁₋₆alkanoyl by reacting the former compound with C₁₋₆alkanoyl chloride in the presence of a base (such as triethylamine) in a suitable solvent (such as a haloalkane, e.g. dichloromethane).

Furthermore, the compound of formula (I) where

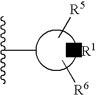

is

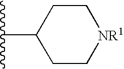

may be converted to the compound of formula (I) where

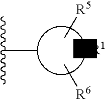

is

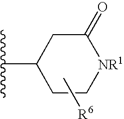

by reacting the former compound with an oxidizing agent (such as KMnO₄) in a suitable solvent (such as a haloalkane, e.g. dichloromethane).

Alternatively, the compound of formula (I) where

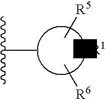

is

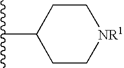

may be converted to the compound of formula (I) where

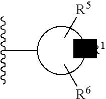

is

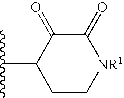

by reacting the former compound with an oxidizing agent (such as sodium periodate), preferably in the presence of a suitable catalyst (such as ruthenium dioxide), in a suitable solvent (such as ethyl acetate). The resultant compound of formula (I) may be further converted to the compound of formula (I) where

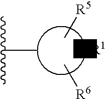

is

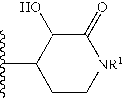

by reacting it with a reducing agent (such as sodium borohydride) in a suitable solvent (such as methanol).

¹H nmr spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are reported in the order: number of protons; multiplicity (s, singlet; d, doublet; t, triplet; q, quartet; m, multiplet; br, broad; and combinations thereof); coupling constant(s) in hertz. Mass spectral (MS) data were obtained on a Waters Micromass ZQ or a Waters Micromass ZMD operating in negative (ES⁻) or positive (ES⁺) ionisation mode and results are reported as the ratio of mass over charge (m/z) for the parent ion only. Preparative scale HPLC separations were carried out using mass triggered HPLC on a preparative Agilent 100 separation module. Compounds were either eluted with linear gradients of acetonitrile/0.1% TFA and water/0.1% TFA or with acetonitrile and water (containing ammonium carbonate to give a pH of 10). In all cases flow rates between 15 and 25 mL/min were used.

Abbreviations used herein, particularly the Schemes and Examples, including the following: DCM, dichloromethane; DMF, dimethyl formamide; DMSO, dimethyl sulfoxide; Et₃N, triethylamine; EtOAc, ethyl acetate; Et₂O, diethyl ether; ES⁺ electrospray; h, hour(s); LHMDS, lithium hexamethyldisilazide; MEOH, methanol; min, minute(s); RT, room temperature; TFA, trifluoroacetic acid; THF, tetrahydrofuran.

The following Descriptions and Examples illustrate the present invention:

Description 1: tert-Butyl 4-[(1E)-3-oxo-3-phenylprop-1-en-1-yl]piperidine-1-carboxylate

To a solution of 4-piperidinemethanol (11.5 g, 0.1 mol) in DCM (200 mL) was added di-t-butyldicarbonate (23.98 g, 0.11 mol) and the mixture was stirred at RT for 16 h. The solvent was removed by evaporation and the resultant solid was dried under vacuum for 3 h.

To a cooled (−60° C.) solution of DMSO (17.2 mL) in DCM (55 mL) was slowly added a solution of oxalyl chloride (10.2 mL) in DCM (140 mL). After stirring the cloudy solution at −60° C. for 20 min, a solution of 1-(t-butoxycarbonyl)-4-piperidinemethanol (prepared above) in DCM (55 mL) was added over 20 min and then the mixture was stirred at −60° C. for an additional 20 min. Et₃N (70 mL) was added and the solution was allowed to warm to RT. Water (100 mL) was added and the organic phase was washed with 1M aqueous citric acid (2×100 mL), water, saturated brine and was dried (MgSO₄). Removal of the solvent by evaporation gave 1-(t-butoxycarbonyl)-4-piperidinecarboxaldehyde as an oil (23.9 g).

To a cooled (−78° C.) solution of 1M-LHMDS in THF (87 mL, 87 mmol) in THF (100 mL) was added a solution of acetophenone (10.44 g, 87 mmol) in THF (30 mL). After stirring the solution at −78° C. for 1 h, a solution of 1-(t-butoxycarbonyl)-4-piperidinecarboxaldehyde (18.6 g, 87 mmol) in THF (50 mL) was added and stirred at −78° C. for 30 min then allowed to warm to -30° C. IM-citric acid (200 mL) was added and the mixture was warmed to RT. EtOAc (400 mL) was added and organic phase was washed with water, brine and was dried (MgSO₄). The solvent was removed under reduced pressure and the residual oil was dissolved in DCM (200 mL), cooled in an ice bath and Et₃N (24.2 mL, 174 mmol) added. Methanesulfonyl chloride (8 mL) was slowly added and the solution was stirred at 0° C. for 60 min and then heated under reflux for 30 min. To the cooled solution was added DCM (200 mL) and saturated aqueous NaHCO₃ and the solution was stirred at RT for 30 min and then separated. The organic phase was dried (MgSO₄) and the solvent was removed under vacuum. The residue was purified by chromatography on silica gel eluting with increasing amounts of EtOAc in isohexane (10-30%). The title compound was obtained as an oil by evaporation (21.6g, 78%). ¹H NMR (500 MHz, CDCl₃): δ 7.92 (2H, t, J 7.1), 7.58-7.46 (3H, m), 6.99 (1H, dd, J 6.5, 15.6), 6.87 (1H, dd, J 1.0, 15.5), 4.14-4.10 (2H, m), 2.80 (2H, m), 2.44-2.38 (1H, m), 1.80 (2H, d, J 12.3), 1.46 (9H, s), 1.46-1.40 (2H, m).

Description 2: tert-Butyl 4-(3-oxo-3-phenylpropyl)piperidine-1-carboxylate
A mixture of the product of Description 1 (21.1 g, 67 mmol) and 10 % Pd on C (1.9 g) dissolved in EtOAc (300 mL) was hydrogenated at 30 psi of H₂ for 6 h. The solution was filtered and evaporated to give the title compound as an oil (20.9 g). ¹H NMR (500 MHz, CDCl₃): δ 7.97-7.91 (2H, d, J 7.8), 7.58-7.52 (1H, t), 7.46 (2H, t, J 7.6), 4.12 (2H, m), 3.00 (2H, t, J 7.5), 2.68 (2H, m), 1.70 (4H, m), 1.42 (10H, m), 1.26 (2H, t, J 7.1).
Description 3: 3-{[1-(tert-Butoxycarbonyl)piperidin-4-yl]methyl}-8-fluoro-2-phenylquinoline-4-carboxylic acid
To a solution of 7-fluoroisatin (8.84 g, 53.6 mmol) in ethanol (55 mL) and aqueous KOH (12 g, 214 mmol dissolved in water (55 mL)) was added the product of Description 2 (17 g, 53.6 mmol). The solution was heated at 100° C. for 5 days under an atmosphere of N₂ then cooled to RT and diluted with water (300 mL). The aqueous phase was washed with Et₂O (2×100 mL), neutralized by addition of acetic acid (8 mL) and the product extracted repeatedly with EtOAc (5×100 mL). The combined EtOAc layers were dried (MgSO₄) and evaporated to foam (11.9 g). This crude product was purified by chromatography on silica eluting with EtOAc/isohexane (1:1) containing 1% AcOH then by 1% AcOH in EtOAc followed by crystallization of the product from EtOAc: isohexane to give the title compound (5.7 g). ¹H NMR (360 MHz, DMSO d₆, 330 K) δ 14.0 (1H, broad s), 7.68-7.48 (8H, m), 3.69 (2H, dm, J 13.3), 2.87 (2H, d, J 7.0), 2.37 (2H, broad t, J 12), 1.4 (1H, m), 1.33 (9H, s), 1.20 (2H, broad d, J 12.9), 0.86-0.72 (2H, m); m/z (ES+) 465 (MH).

### EXAMPLE 1

### (S)-tert-Butyl 4-[(8-fluoro-2-phenyl-4-{[(1-phenylpropyl)amino]carbonyl}quinolin-3-yl)methyl]piperidine-1-carboxylate

To a cooled (0° C.) solution of DMF (10 mL) in DCM (100 mL) was slowly added oxalyl chloride (0.776 mL, 8.9 mmol). The solution was stirred at this temperature for an additional 30 min after the effervescence had subsided when the product of Description 3 (2.0 g, 4.34 mmol) was added and the solution was stirred at 0° C. for 2 h. To this was added a solution of Et₃N (2 mL, 14.4 mmol) and (S)-(−)-1-phenylpropylamine (0.586 mg, 4.34 mmol). The solution was stirred at RT for 16 h, evaporated to dryness and the residue partioned between EtOAc and saturated NaHCO₃. The organic phase was washed with water (4 times), saturated brine and was dried (MgSO₄). After evaporation of the solvent, the residue was purified by chromatography on silica eluting with increasing concentrations of EtOAc in isohexane (0% -20%) to give the title compound 1.54 g. ¹H NMR (500 MHz, CDC1₃): δ 7.49-7.30 (13H, m), 6.15 (1H, d, J 8.3), 5.22 (1H, m), 4.00-3.5 (2H, broad m), 3.14-2.12 (4H, broad m), 2.11-1.90 (2H, m), 1.40 (10H, m), 1.1-0.8 (6H, m), 0.71-0.37 (1H, m). m/z (ES+) 582 (MH)

### EXAMPLE 2

### (S)-8-Fluoro-2-phenyl-N-(1-phenylpropyl)-3-(piperidin-4-ylmethyl)quinoline-4-carboxamide

The product of Example 1 (1.54 g, 2.65 mmol) was dissolved in anhydrous TFA (10 mL). After 30 min, the solvent was removed by evaporation and the residue was partitioned between EtOAc and 5% aqueous NaHCO₃ solution. The organic phase was dried (MgSO₄) and evaporated to give the title compound as a foam. ¹H NMR (500 MHz, MeOH d4) δ 7.77 (0.6H, d, J 8.4), 7.69-7.29 (12H, m), 7.15 (0.4H, d, J 8.4), 5.15 (0.6H, t, J 7.3), 5.09 (0.4H, t, J 7.6), 3.15 (1H, m), 2.94 (1.6H, m), 2.81 (0.6H, m), 2.68 (0.4H, td), 2.61 (0.4H, td), 2.5 (0.6H, m), 2.4 (1.4H, m), 2.05-1.88 (2.4H, m), 1.71-1.58 (0.6H, m), 1.49 (0.4H, d), 1.37 (0.6H, m), 1.18-0.95 (4.4H, m), 0.8-0.71 (1.6H, m). m/z (ES+) 482 (MH).

### EXAMPLE 3

### (S)-8-Fluoro-2-phenyl-N-(1-phenylpropyl)-3-{[1-(tetrahydro-2H-pyran-4-yl)piperidin-4-yl]methyl}quinoline-4-carboxamide

To a solution of the product of Example 2 (1.30 g, 2.65 mmol), tetrahydro-4H-pyran-4-one (1.22 mL, 13.25 mmol) and acetic acid (0.16 mL) in 1,2-dichloroethane (10 mL) was added sodium triacetoxyborohydride (2.81 g, 13.25 mmol). The solution was stirred at RT for 16 h then 1M aqueous HCl (30 mL) was added and stirring continued for 30 min. The solution was neutralized by the addition of solid NaHCO₃ and the product was extracted with EtOAc (2×50 mL). The combined organic phases were dried (MgSO₄), evaporated to dryness and the residue purified by chromatography on silica by elution with 20%, 50%, 100% EtOAc in isohexane followed by 2% and 10% MeOH in EtOAc. The product was purified further by chromatography on silica eluting with 0%, 1% and 5% MeOH in DCM. After evaporation of the solvent 1M-HCl in Et₂O (1.88 mL) was added to an EtOAc solution of the residue and the evaporated and dried in vacuo to give the title compound. ¹H NMR (700 MHz, MeOH d₄) (rotational isomers) δ 7.76 (0.66H, d, J 8.4), 7.67 (0.74H, m), 7.6-7.3 (11.34H, m), 7.16 (0.3H, d, J 8.3), 5.14 (0.64H, t, J 7.5), 5.09 (0.36H, t, J 7.4), 4.0 (2H, m), 3.46-3.18 (5H, m), 2.92-2.83 (1H, m), 2.75-2.43 (3H, m), 2.00-1.83 (4H, m), 1.75-1.57 (3H, m), 1.34 (2H, m), 1.21-1.04 (4H, m), 0.91 (0.55H, m), 0.79 (0.45H, m). m/z (ES+) 566 (MH).
Description 4: 3-{[1-(tert-Butoxycarbonyl)piperidin-4-yl]methyl}-2-phenylquinoline-4-carboxylic acid
The title compound was prepared in an analogous manner to that described in Description 3 using isatin and the product of Description 2.

### EXAMPLE 4

### (S)-tert-Butyl 4-[(2-phenyl-4-{[(1-phenylpropyl)amino]carbonyl}quinolin-3-yl)methyl]piperidine-1-carboxylate

To a cooled (0° C.) solution of DCM (5 mL) and DMF (0.5 mL) was added oxalyl chloride 0.038 mL). After 30 mins a solution of the product of Description 4 (100 mg, 0.224 mmol) in DCM (5 mL) was added. After stirring the solution at 0° C. for 1.5 h the solution was evaporated to a small volume and the residue was quickly partioned between EtOAc and NaHCO₃ solution and the organic phase dried (MgSO₄) and evaporated to dryness. The residue was dissolved in THF (20 mL) together with (S)(−)-1-phenylpropylamine (81.5 mg, 0.602 mmol) and the solution was stirred at RT for 16 h. The solution was evaporated to dryness and the residue partitioned between EtOAc and aqueous citric acid. The organic phase was washed successively with NaHCO₃, water, brine and was dried (MgSO₄). The residue, after evaporation, was purified by silica chromatography eluting with 10-40% EtOAc in hexane to give the title compound as a foam; m/z (ES+) 564 (MH).

### EXAMPLE 5

### (S)-4-[(2-phenyl-4-([(1-phenylpropyl)amino]carbonylquinolin-3-yl)methyl]piperidine

The product of Example 4 (113 mg) was dissolved in TFA (5 mL) and after 30 mins the solvent was removed in vacuo and the residue partitioned between EtOAc and NaHCO₃. The organic phase was dried (MgSO₄) to give the title compound as a foam 96 mg; m/z (ES+) 464 (MH)

### EXAMPLE 6

### (R)-4-[(2-phenyl-4-{[(1-phenylpropyl)amino]carbonyl}quinolin-3-yl)methyl]piperidine

The title compound was prepared by coupling (R) 1-phenylpropylamine to the product of Description 4 followed by deprotection by procedures analogous to that described in Example 4 and Example 5.

### EXAMPLE 7

### (S)-Benzyl-4-[(2-phenyl-4-{[(1-phenylpropyl)amino]carbonyl}quinolin-3-yl)methyl]piperidine-1-carboxylate

The product of Example 5 (39 mg) was dissolved in a mixture of EtOAc (2 mL) and saturated NaHCO₃ (2 mL) together with benzylchloroformate (0.017 mL). The solution was stirred at RT for 4 h, then EtOAc (20 mL) was added and the organic phase was dried (MgSO₄). After evaporation the residue was purified by silica gel chromatography eluting with 10%-40% EtOAc in hexane to give the title compound; m/z (ES+) 598 (MH).

### EXAMPLE 8

### (S)-4-[(2-phenyl-4-{[(1-phenylpropyl)amino]carbonyl}quinolin-3-yl)methyl]-1-(tetrahydropyran-4-yl)piperidine

To a solution of the product of Example 5 (37 mg, 0.0799 mmol) in dichloroethane (2 mL) was added tetrahydro-4H-pyran-4-one (0.15 mL), acetic acid 0.004 mL), sodium triacetoxyborohydride (0.15 g) and the solution was stirred at RT for 20 h. To the solution was added 2M-HCl (2 mL) and after 30 mins water was added together with sufficient solid NaHCO₃ until pH 7. DCM was added and the product was extracted. The organic phase was dried (MgSO₄), evaporated and the residue purified by chromatography on silica gel eluting with DCM to 4% MeOH in DCM to give the title compound (26 mg); m/z (ES+) 548 (MH).

### EXAMPLE 9

### (R)-4-[(2-phenyl-4-{[(1-phenylpropyl)amino]carbonyl}quinolin-3-yl)methyl]-1-(tetrahydropyran-4-yl)piperidine

The title compound was prepared from the product of Example 6 by a procedure analogous to that described in Example 8 to give the title compound; m/z (ES+) 548 (MH).
Description 5: rac tert-Butyl 3-[(1E)-3-oxo-3-phenylprop-1-en-1-yl]piperidine-1-carboxylate
To a solution of 3-piperidinemethanol (11.5 g, 0.1 mol) in DCM (200 mL) was added di-t-butyldicarbonate (23.98 g, 0.11 mol) and the mixture was stirred at RT for 16 h. The solvent was removed by evaporation and the resultant solid was dried under vacuum for 3 h. to yield 1-t-butyloxycarbonyl-3-piperidinemethanol (23.1 g)

To a cooled (−60° C.) solution of DMSO (17.2 mL) in DCM (55 mL) was slowly added a solution of oxalyl chloride (10.2 mL) in DCM (140 mL). After stirring the cloudy solution at −60° C. for 20 min, a solution of 1-t-butoxycarbonyl-3-piperidinemethanol (prepared above) in DCM (55 mL) was added over 20 min and then the mixture was stirred at −60° C. for an additional 20 min. Et₃N (70 mL) was added and the solution was allowed to warm to RT. Water (100 mL) was added and the organic phase was washed with 1M aqueous citric acid (2×100mnL), water, saturated brine and was dried (MgSO₄). Removal of the solvent by evaporation gave 1-(t-butoxycarbonyl)-3-piperidinecarboxaldehyde as an oil (22.2 g).

To a cooled (−78° C.) solution of 1M-LHMDS in THF (100 mL, 100 mmol) in THF (50 mL) was added a solution of acetophenone (12 g, 100 mmol) in THF (50 mL). After stirring the solution at −78° C. for 0.5 h, a solution of 1-(t-butoxycarbonyl)-3-piperidinecarboxaldehyde (22 g, 98 mmol) in THF (50 mL) was added and stirred at −78° C. for 30 min then allowed to warm to −30° C. IM-citric acid (200 mL) was added and the mixture was warmed to RT. EtOAc (400 mL) was added and organic phase was washed with water, brine and was dried (MgSO₄). The solvent was removed under reduced pressure and the residual oil was dissolved in DCM (100 mL), cooled in an ice bath and Et₃N (27.8 mL, 200 mmol) added. Methanesulfonyl chloride (7.7 mL) was slowly added and the solution was stirred at 0° C. for 120 min. To the cooled solution was added DCM (100 mL) and saturated aqueous NaHCO₃ (100 mL) and the solution was stirred at RT for 30 min and then separated. The organic phase was dried (MgSO₄) and the solvent was removed under vacuum. The residue was purified by chromatography on silica gel eluting with increasing amounts of EtOAc in isohexane (10-15%). The title compound was obtained as an oil by evaporation (15.3 g, 50%). ¹H NMR (500 MHz, CDCl₃): δ 7.93 (2H, d, J 8.4), 7.58 (1H, t J 7.7), 7.47 (2H, t, J 7.7), 6.94 (2H, m), 4.05-3.85 (2H, m), 2.85 (2H, m), 2.45 (1H, m), 1.95 (1H, m), 1.7 (1H, m), 1.55-1.4 (11H, m).

Description 6: rac tert-Butyl 3-(3-oxo-3-phenylpropyl)piperidine-1-carboxylate
A mixture of the product of Description 5 (3.4 g, 10.8 mmol) and 10 % Pd on C (0.37 g) dissolved in EtOAc (100 mL) was hydrogenated at 30 psi of H₂ for 4 h. The solution was filtered and evaporated to give the title compound as an oil (3.5 g). ¹H NMR (500 MHz, CDCl₃): δ 7.95 (2H, d, J 8.5), 7.58-7.52 (1H, t J 7.4), 7.46 (2H, t, J 7.6), 3.9 (1H, dm), 3.03 (2H, m), 2.8 (1H, tm), 2.5 (1H, broad m), 1.87 (1H, m), 1.67 (4H, m), 1.46 (llH, m), 1.15 (1H, m).
Description 7: rac 3-{[1-(tert-Butoxycarbonyl)piperidin-4-yl]methyl}-8-fluoro-2-phenylquinoline-3-carboxylic acid
To a solution of 7-fluoroisatin (1.81 g, 11.0 mmol) in ethanol (11 mL) and aqueous KOH (1.45 g, 43.8 mmol dissolved in water (11 mL)) was added the product of Description 6 (3.47 g, 11 mmol). The solution was heated at 100° C. for 3 days under an atmosphere of N₂ then cooled to RT and diluted with water (30 mL). The aqueous phase was washed with Et₂O (2×30 mL), neutralized by addition of acetic acid (2.6 mL) and the product extracted repeatedly with EtOAc (5×20 mL). The combined EtOAc layers were dried (MgSO₄) and evaporated to a solid (1.66 g). ¹H NMR (400 MHz, DMSO d6, 299 K) δ very broad spectrum, single peak by analytical hplc; m/z (ES+) 465 (mH).

### EXAMPLE 10

### tert-Butyl 3-[(8-fluoro-2-phenyl-4-{[((S)-1-phenylpropyl)amino]carbonyl}quinolin-3-yl)methyl]piperidine-1-carboxylate

To a cooled (0° C.) solution of DMF (5 mL) and DCM (50 mL) was added oxalyl chloride (0.413 mL, 4.74 mmol). After 30 mins at 0° C., a solution of the product of Description 7 (1.1 g, 2.11 mmol) dissolved in DCM (30 mL) was added for a further 45 mins. TEA (1.32 mL, 9.48 mmol) was added followed by (S)(−)-1-phenylpropylamine (0.61 g, 4.52 mmol) dissolved in DCM (5 mL). After stirring for 1 h at 0° C. the solution was evaporated and to the residue was added EtOAc and aqueous 10% citric acid solution. The organic phase was washed with water (×3) and brine (×1) and was dried (MgSO₄). The solution was evaporated to dryness and the residue was purified by chromatography on silica gel eluting with 5-20% EtOAc in hexane to give the title compound 1.32 g as an inseparable mixture of diastereomers by hplc and tlc; m/z (ES+) 583 (MH).

### EXAMPLE 11

### 3-[(8-fluoro-2-phenyl-4-{[((S)-1-phenylpropyl)amino]carbonyl}quinolin-3-yl)methyl]piperidine

The product of Example 10 (1.22 g) was treated with TFA (20 mL) and after 20 mins the solution was evaporated to dryness and the residue was partitioned between EtOAc and saturated NaHCO₃. The organic phase was dried (MgSO₄) and then evaporated to a foam to give the title compound 1.28 g as a mixture of diastereomers; m/z (ES+) 482 (MH).

### EXAMPLES 12 AND 13

### 3-(R or S)-[(8-fluoro-2-phenyl-4-{[((S)-1-phenylpropyl)amino]carbonyl}quinolin-3-yl)methyl]-1-(tetrahydropyran-4-yl)piperidine and 3-(S or R)-[(8-fluoro-2-phenyl-4-{[((S)-1-phenylpropyl)amino]carbonyl}quinolin-3-yl)methyl]-1-(tetrahydropyran-4-yl)piperidine

To a solution of the product of Example 11 (0.173 g, 0.360 mmol) in DCM (5 mL) was added tetrahydro-4H-pyran-4-one (0.166 mL, 1.798 mmol), acetic acid 0.022 mL), sodium triacetoxyborohydride (0.381 g, 1.80 mmol) and the solution was stirred at RT for 16 h. To the solution was added 2M-HCl (5 mL) and after 30 mins solid NaHCO₃ was added until pH 7 with addition of water and the product was extracted by addition of DCM. The organic phase was dried (MgSO₄), evaporated and the residue purified by chromatography on silica gel eluting with EtOAc followed by 2% MeOH in EtOAc to give the title compound as a mixture of diastereomers (134 mg) m/z (ES+) 567 (MH). The diastereomer mixture was separated into individual diastereomers by super critical fluid (SFC) chromatography to give
Diastereomer A (first eluting) m/z (ES+) 567 (MH).
Diastereomer B (later eluting) m/z (ES+) 567 (MH).

### EXAMPLE 14

### 4-[(2-phenyl-4-{[((S)-1-phenylpropyl)amino]carbonyl}quinolin-3-yl)methyl]-1-(tetrahydropyran-4-yl)piperidin-2-one

To a solution of the product of Example 8 (89 mg) and benzyltriethylammonium chloride (100 mg) dissolved in DCM (4 mL) was added KMnO₄ (100 mg). The solution was stirred at RT for 10 mins when water (10 mL) and solid sodium metabisulphite added until colourless. The solution was diluted by addition of EtOAc and water and the organic phase was dried (MgSO₄). After evaporation to dryness the residue was purified by preparative hplc to give the title compound 22 mg; m/z (ES+) 563 (MH).

### EXAMPLE 15

### 4-[(2-phenyl-4-{[((S)-1-phenylpropyl)amino]carbonyl}-8-fluoro-quinolin-3-yl)methyl]-1-(tetrahydropyran-4-yl)piperidin-2-one

The title compound was prepared in a manner analogous to that described in Example 14 using as starting material the product of Example 3 to give the title compound; m/z (ES+) 580 (MH).

### EXAMPLE 16

### 4-[(2-phenyl-4-{[((S)-1-phenylpropyl)amino]carbonyl}quinolin-3-yl)methyl]-1-(tetrahydropyran-4-yl)piperidin-2,3-dione

The product of Example 8 (114 mg, 0.21 mmol) and sodium periodate (260 mg, 1.22 mmol) were dissolved in EtOAc (9 mL) and water (9 mL) and to the stirred solution was added catalytic ruthenium dioxide monohydrate (0.05 mg). The solution was stirred at RT for 3 h (after following the reaction by mass spec). The solution was separated and the organic phase was washed with water (×3), 5% sodium metabisulfite solution and was then dried (MgSO₄). After removal of the solvent by evaporation the residue was purified by preparative hplc to give the title compound 23 mg; m/z (ES+) 577 (MH). ¹H NMR exists as a mixture of keto and enol tautomers.

### EXAMPLE 17

### 4-[(2-phenyl-4-([((S)-1-phenylpropyl)amino]carbonyl)-8-fluoro-quinolin-3-yl)methyl]-1-(tetrahydropyran-4-yl)piperidin-2,3-dione

The title compound was prepared in a manner analogous to that described in Example 16 using as starting material the product of Example 3; m/z (ES+) 594 (MH).

### EXAMPLE 18

### S-4-[(2-phenyl-4-{[(1-phenylpropyl)amino]carbonyl}-8-fluoro-quinolin-3-yl)methyl]-(1-methylethyl)-piperidine

To a solution of the product of Example 3 (0.1 g, 0.21 mmol) in DCE (5 mL), acetic acid (0.012 mL) and acetone (0.076 mL) was added sodium triacetoxyborohydride (0.218 g, 1.03 mmol). The solution was stirred at RT for 72 h then DCM (15 mL) and sat NaHCO₃ (15 mL) were added and the organic phase was dried (MgSO₄). After evaporation the title compound was isolated by preparative hplc; m/z (ES+) 524 (MH).

### EXAMPLE 19

### S-4-[(2-phenyl-4-{[(1-phenylpropyl)amino]carbonyl}-8-fluoro-quinolin-3-yl)methyl]-1-(methylsulphonyl)-piperidine

To a cooled (0° C.) solution of the product of Example 3 (0.2 g, 0.42 mmol) and TEA (1.25 mmol) dissolved in DCM (5 mL) was added methanesulfonyl chloride (0.065 mL). After 1 h a solution of NaHCO₃ was added and the organic phase was washed with 1M-HCl, brine and dried (MgSO₄). After evaporation the residue was purified by silica chromatography eluting with 20-35% EtOAc in hexane to give the title compound; m/z (ES+) 558(MH).

### EXAMPLE 20

### S-4-[(2-phenyl-4-{[(1-phenylpropyl)amino]carbonyl}-8-fluoro-quinolin-3-yl)methyl]-1-(pyridine-3-yl)-piperidine

To a suspension of pyridine-3-boronic acid (0.051 g, 0.416 mmol), copper(II)acetate (0.0083 g, 0.0416 mmol) and powdered A4 molecular sieves (0.155 g) was added the product of Example 3 (0.1 g, 0.21 mmol) and the mixture heated to 40° C. for 16 h. The solution was cooled, diluted with DCM and water and the organic phase was purified by preparative hplc to give the title compound; m/z (ES+) 559 (MH).

### EXAMPLE 21

### S-4-[(2-phenyl-4-{[(1-phenylpropyl)amino]carbonyl}-8-fluoro-quinolin-3-yl)methyl]-1-(2-methylpropanoyl)-piperidine

To a solution of the product of Example 3 (0.1 g, 0.21 mmol) and TEA (0.02 g) in DCM (2 mL) was added isobutyryl chloride. After stirring the solution at RT for 16 h, water was added and the organic phase was washed with water, brine and was dried (MgSO₄). After evaporation the product was purified by chromatography on silica gel to give the title compound m/z (ES+) 552 (MH).

### EXAMPLE 22

### 4-[(2-phenyl-4-{[((S)-1-phenylpropyl)amino]carbonyl}-8-fluoro-quinolin-3-yl)methyl]-3-(R or S)-hydroxy-1-(tetrahydropyran-4-yl)piperidin-2-one and Example 23: 4-[(2-phenyl-4-{[((S)-1-phenylpropyl)amino]carbonyl}-8-fluoro-quinolin-3-yl)methyl]-3-(S or R)-hydroxy-1-(tetrahydropyran-4-yl)piperidin-2-one

To a solution of the equilibrated product of Example 17 (40 mg) in MeOH (5 mL) was added solid NaBH₄ (approximately 50 mg) in portions until starting material was absent by hplc. 1M-HCl (2 mL) was added and when the effervescence had stopped EtOAc and water were added. The organic phase was separated and dried (MgSO₄). After evaporation of the solvent the residue dissolved in DMSO (1 mL) was purified into two separate diastereomers using mass-triggered hplc to give diastereomer 1 (19 mg) (m/z (ES+) 596 (MH) and diastereomer 2 (8.1 mg) m/z (ES+) 596 (MH).

1-19. (canceled)

20. A compound of the formula (I): 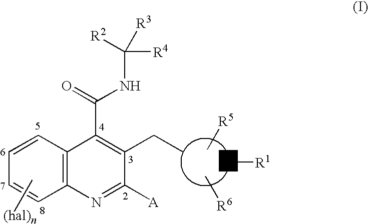
wherein:
hal is fluorine, chlorine, bromine or iodine;
n is 0, 1 or 2, and when n is 2, the two hal atoms may be the same or different;
A is phenyl or thiophenyl, which is unsubstituted or substituted with 1 to 3 halogen atoms; 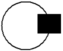
is a C-linked azetidinyl, pyrrolidinyl or piperidinyl ring, optionally bridged by a C₁₋₃alkylene group, and optionally fused to phenyl;
R¹ is hydrogen, C₁₋₆alkyl, C₂₋₆alkenyl, C₂₋₆alkynyl, C₃₋₈cycloalkyl, C(O)C₁₋₆alkyl, C(O)OC₁₋₆alkyl, C(O)O(CH₂)₀₋₃aryl, S(O)₂C₁₋₆alkyl, heteroaryl or Het, where C₃₋₈cycloalkyl, aryl, heteroaryl and Het are unsubstituted or substituted with C₁₋₆alkyl, and where Het is a heteroaliphatic ring of 4 to 6 ring atoms, which ring contains 1 or 2 heteroatoms selected from N, O and S or a group S(O), S(O)₂, NH or NC₁₋₄alkyl;
R² is hydrogen, C₁₋₆alkyl, C₂₋₆alkenyl, C₂₋₆alkynyl or C₃₋₈cycloalkyl;
R³ is C₁₋₆alkyl, C₂₋₆alkenyl, C₂₋₆alkynyl, (CH₂)₀₋₃C₃₋₈cycloalkyl or (CH₂)₀₋₃phenyl, which is unsubstituted or substituted with 1 to 3 halogen atoms;
R⁴ is hydrogen, C₁₋₆alkyl, C₂₋₆alkenyl, C₂₋₆alkynyl or C₃₋₈cycloalkyl;
or R² and R⁴ are linked together to form a C₃₋₈cycloalkyl or Het group;
R⁵ is hydrogen, C₁₋₆alkyl, C₂₋₆alkenyl, C₂₋₆alkynyl, C₃₋₈cycloalkyl or oxo;
or R¹ and R⁵ together form a nitrogen-containing heteroaliphatic ring, optionally containing one further N or O atom, and optionally substituted by C₁₋₆alkyl;
R⁶ is hydrogen, hydroxy or oxo; with the proviso that when R⁶ is hydroxy it is not attached to the carbon atom adjacent to the ring N atom;
or a pharmaceutically acceptable salt thereof.

21. The compound of claim 20 of formula (Io): 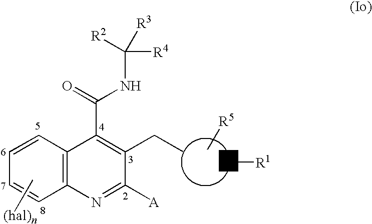
wherein:
hal is fluorine, chlorine, bromine or iodine;
n is 0, 1 or 2, and when n is 2, the two hal atoms may be the same or different;
A is phenyl or thiophenyl, which is unsubstituted or substituted with 1 to 3 halogen atoms; 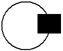
is a C-linked azetidinyl, pyrrolidinyl or piperidinyl ring, optionally bridged by a C₁₋₃alkylene group, and optionally fused to phenyl;
R¹ is hydrogen, C₁₋₆alkyl, C₂₋₆alkenyl, C₂₋₆alkynyl, C₃₋₈cycloalkyl or Het, where C₃₋₈cycloalkyl and Het are unsubsituted or substituted with C₁₋₆alkyl, and where Het is a heteroaliphatic ring of 4 to 6 ring atoms, which ring contains 1 or 2 heteroatoms selected from N, O and S or a group S(O), S(O)₂, NH or NC₁₋₄alkyl;
R² is hydrogen, C₁₋₆alkyl, C₂₋₆alkenyl, C₂₋₆alkynyl or C₃₋₈cycloalkyl;
R³ is C₁₋₆alkyl, C₂₋₆alkenyl, C₂₋₆alkynyl, (CH₂)₀₋₃C₃₋₈cycloalkyl or (CH₂)₀₋₃phenyl, optionally substituted by 1 to 3 halogen atoms;
R⁴ is hydrogen, C₁₋₆alkyl, C₂₋₆alkenyl, C₂₋₆alkynyl or C₃₋₈cycloalkyl;
or R² and R⁴ are linked together to form a C₃₋₈cycloalkyl or Het group as hereinbefore defined;
R⁵ is hydrogen, C₁₋₆alkyl, C₂₋₆alkenyl, C₂₋₆alkynyl or C₃₋₈cycloalkyl;
or R¹ and R⁵ together form a nitrogen-containing heteroaliphatic ring, optionally containing one further N or O atom, and optionally substituted by C₁₋₆alkyl.

22. The compound of claim 20 wherein hal is fluorine, chlorine or bromine.

23. The compound of claim 20 wherein n is 1 or 2.

24. The compound of claim 20 wherein A is phenyl, which is unsubstituted or substituted 1 or 2 halogen atoms.

25. The compound of claim 20 wherein 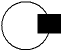
is a C-linked pyrrolidinyl or piperidinyl ring.

26. The compound of claim 20 wherein R¹ is C₁₋₆alkyl or Het, where Het is unsubstituted or substituted with C₁₋₆alkyl.

27. The compound of claim 20 wherein R² is C₁₋₆alkyl.

28. The compound of claim 20 wherein R³ is C₁₋₆alkyl, C₃₋₈cycloalkyl or (CH₂)₀₋₃phenyl.

29. The compound of claim 20 wherein R⁴ is hydrogen or C₁₋₆alkyl.

30. The compound of claim 20 wherein R⁵ is hydrogen or C₁₋₆alkyl.

31. The compound of claim 20 of the formula (Ia): 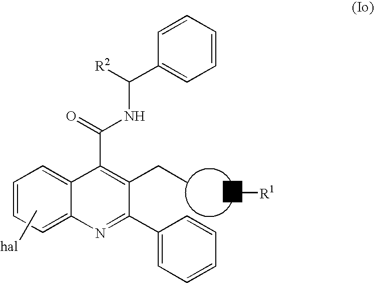
or a pharmaceutically acceptable salt thereof.

32. The compound of claim 20 of the formula (Ib): 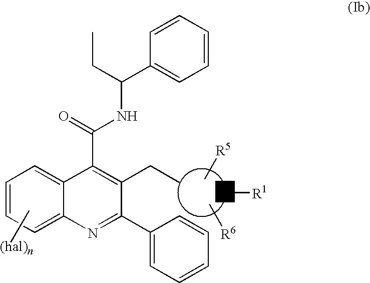
or a pharmaceutically acceptable salt thereof.

33. A compound which is selected from:
(S)-8-fluoro-2-phenyl-N-(1-phenylpropyl)-3-(piperidin-4-ylmethyl)quinoline-4-carboxamide,
(S)-8-fluoro-2-phenyl-N-(1-phenylpropyl)-3-{[1-(tetrahydro-2H-pyran-4-yl)piperidin-4-yl]methyl}quinoline-4-carboxamide,
or a pharmaceutically acceptable salt thereof.

34. A pharmaceutical composition comprising the compound of claim 20 or a pharmaceutically acceptable salt thereof and a pharmaceutically acceptable excipient.

35. A method for the treatment of a subject suffering from a neurokinin-2 and/or neurokinin-3 mediated disease, which comprises administering to that patient a therapeutically effective amount of the compound of claim 20 or a pharmaceutically acceptable salt thereof.

36. The method of claim 35 wherein the neurokinin-2 and/or neurokinin-3 mediated disease is selected from the group consisting of: anxiety disorder; phobia; psychosis; psychotic disorder; post-traumatic stress disorder; attention deficit hyperactive disorder (ADHD); withdrawal from abuse of drugs including smoking cessation or reduction in level or frequency of such activities; and irritable bowel syndrome.
